(19) United States
(12) Patent Application Publication
(10) Pub. No.: US 2020/0109388 A1
(43) Pub. Date: Apr. 9, 2020

(54) Recovery Process

(71) Applicant: Novozymes A/S, Bagsvaerd (DK)

(72) Inventors: Simon Glanville, Kalundborg (DK); Peter Frode Pind, Herlev (DK); Sune Jakobsen, Vaerloese (DK); Lars Johansen, Bagsvaerd (DK); Carsten Jacobsen, Copenhagen (DK); Kim Bruno Andersen, Vaerloese (DK); Søren Prip Beier, Bagsvaerd (DK); Jens-Ulrik Rype, Smoerum (DK)

(73) Assignee: Novozymes A/S, Bagsvaerd (DK)

(21) Appl. No.: 16/500,786

(22) PCT Filed: Apr. 3, 2018
(86) PCT No.: PCT/EP2018/058387

§ 371 Date: Oct. 3, 2019

(30) Foreign Application Priority Data
Apr. 3, 2017 (EP) 17164452.9

(51) Int. Cl. C12N 9/54; C12N 9/36

(52) U.S. Cl. CPC C12N 9/54; C12Y 302/01017; C12Y 304/21062; C12N 9/2462

(57) ABSTRACT

Disclosed is a method for recovering a desired fermentation product from a fermentation broth where the desired product has precipitated during the fermentation.

### REFERENCE TO SEQUENCE LISTING

This application contains a Sequence Listing in computer readable form. The computer readable form is incorporated herein by reference.

### FIELD OF THE INVENTION

The present invention relates to an improved method for solubilizing protein crystals and/or protein precipitate in a fermentation broth.

### BACKGROUND OF THE INVENTION

The fermentation yield of industrial proteins, e.g. enzymes such as proteases, has increased dramatically in the recent years. In many industrial processes, the concentration of the protein in the fermentation broth is so high that a significant part of the protein is found in form of crystals or solid precipitates at the end of the fermentation process.

WO93/13125 discloses a process for producing protease where the protease is precipitated during fermentation by adding a precipitating agent to the production medium. It was found that precipitating protease during fermentation protects the protease against proteolysis.

For many fermentation products it is desired that the microorganism used in fermentation is removed from the product. One of the first steps in the product recovery process is usually removal of the cellular biomass from the product, for example by filtration or centrifugation removing the solid cellular biomass from the liquid comprising the desired product. This step is often called the primary separation step where the microorganism is separated from the liquid fraction, and the liquid fraction can subsequently be processed with one or more downstream steps, such as ultrafiltration, stabilisation, spray drying, granulation which eventually will turn the desired product into the form and concentration that is intended for this particular product.

However, if part of the desired product is present in solid form after the fermentation, suitable measures must be taken to solubilize the product before the biomass is removed from the liquid fraction comprising the product.

U.S. Pat. No. 6,316,240 discloses a process for solubilizing enzymes precipitated during fermentation process where the pH of the culture broth is adjusted to a pH between 9.5 and 13.0. The process is exemplified with an amylase fermentation.

EP 2 125 865 discloses a process for solubilizing protease crystals and/or protease precipitate in a fermentation broth comprising diluting the fermentation broth 100-2000% (w/w); adding a divalent salt; and adjusting the pH value of the fermentation broth to a pH value below 5.5.

EP 1 456 613 discloses a process for harvesting crystalline particles, in particular crystalline alpha-amylase or protease, from fermentation broth, wherein the fermentation broth is separated into a biomass fraction, a crystalline alpha-amylase or protease fraction and a supernatant fraction.

### SUMMARY OF THE INVENTION

The invention provides a method for recovering a desired product from a fermentation broth, where the desired product is present in precipitated form in the fermentation broth, the method comprising:
a) a first separation step separating the fermentation broth in a first phase and a second phase, wherein the first phase comprises supernatant from the fermentation broth and it may comprise a part of the cells and cell debris from the fermentation broth, and the second phase comprises the desired product in precipitated form, cells and cell debris; and
b) a solubilisation step where the desired product in precipitated form is solubilized.

In one preferred embodiment the method further comprise a second separation step where the solubilized desired product from step b) is separated from the cells and/or cell debris.

### DETAILED DESCRIPTION OF THE SEQUENCES

SEQ ID NO: 1: amino acid sequence of B. lentus protease (Savinase)

SEQ ID NO: 2: amino acid sequence of B. amyloliquefaciens protease (BPN′)

SEQ ID NO: 3: Subtilisin Carlsberg

SEQ ID NO: 4: Protease from Bacillus sp. TY145, disclosed in WO 92/17577

SEQ ID NO: 5: Protease from Nocardiopsis sp. NRRL 18262, disclosed in WO 88/03947

SEQ ID NO: 6: Pyrococcus furiosus protease, disclosed in U.S. Pat. No. 6,358,726.

SEQ ID NO: 7: Alpha-amylase from Bacillus sp. disclosed in WO 2000/060060.

SEQ ID NO: 8: Alpha-amylase from Bacillus stearothermophilus SEQ ID NO: 9: Alpha-amylase from Bacillus licheniformis

### SHORT DESCRIPTION OF THE DRAWINGS

FIG. 1 shows the dissolution of muramidase from culture broth (CB) or from the second phase according to the invention, reflecting the results of the experiments in example 5.

### DETAILED DESCRIPTION OF THE INVENTION

The present invention relates to the primary separation of a fermentation broth where part of the desired fermentation product is present in solid form such as in crystalline or amorphous form. It is not important for the present invention whether the desired fermentation product is in crystalline or amorphous form or even in a mixture of these forms, what is important is that a significant part of the desired product is in solid form and therefore can the primary separation step not readily be performed as a simple solid/liquid separation process. The primary separation step has the purpose of removing the cells and cell debris from the desired product.

The invention relates to a method of recovering a desired fermentation product from a fermentation broth, wherein the fermentation product is present in at least partially in insoluble form in the fermentation broth such as in crystalline or amorphous form; comprising:
a first separation step separating the fermentation broth in a first phase and a second phase, wherein the first phase comprising liquid from the fermentation broth and desired fermentation product in soluble form; and the second phase comprises the desired fermentation product in insoluble form, cells and/or cell debris; and
a solubilisation step, wherein the desired fermentation product in the second phase is solubilized.

In some embodiments the invention relates to a method further comprising a second separation step separating the liquid phase comprising of the solubilized desired fermentation product from the solid biomass, optionally after mixing the solubilized first phase with part or all of the first phase from the first separation step.

The invention is based on the observation that fermentation broth appears to contain some components that are detrimental to the solubilisation process. Data seems to indicate that optimal conditions for high solubility for proteins such as enzymes, are conditions with high enzyme concentration and low ion concentration. However, it should be understood that the invention is not limited by any particular theory. It has been found beneficial if the solubilisation of the desired product can take place in the absence of at least part of the liquid part of the fermentation broth containing the majority of these disadvantageous components.

According to the invention fermentation is intended to mean the process where one or more microorganisms are grown in a fermenter in a fermentation medium comprising all component necessary for the growth of the one or more microorganisms producing a fermentation broth comprising the one or more microorganisms, spent substrate components and one or more desired fermentation products. If the yield of the desired fermentation product is sufficient high, part of the fermentation product may precipitate forming crystals or amorphous precipitates in the fermentation broth. This is well known in the art and many fermentation processes, microorganisms, medium components have been disclosed in the art. The invention is not limited by the particular fermentation process as long as the fermentation process provides a fermentation broth comprising a desired fermentation product at least partially in insoluble form in the fermentation broth such as in crystalline or amorphous form.

Methods for fermenting microorganism for producing a desired product where the desired product precipitates during the fermentation has been described in the art e.g. U.S. Pat. No. 6,316,240, WO 03/050274, WO 93/13125 and EP2125865 and any of these methods for fermenting the microorganisms can be used together with the methods of the present invention.

The fermentation process may be any suitable fermentation process providing a fermentation broth comprising a desired fermentation product at least partially in insoluble form, such as a batch fermentation, fed-batch or continuous fermentation.

The present invention may be useful for any fermentation in industrial scale, e.g. for any fermentation having culture media of at least 50 liters, such as at least 500 liters, such as at least 5,000 liters, such as at least 50,000 liters.

The fermentation product may be any product produced by microorganisms accumulating in the fermentation broth. The fermentation product may be primary metabolites, secondary metabolites, proteins, vitamins, hormones and carbohydrates. The fermentation product is preferably selected among proteins, such as enzymes. The fermentation product may even be a mixture of two or more desired enzymes e.g. a mixture comprising all enzymes necessary to degrade cellulose into low molecular weight sugars, such as glucose and cellubiose.

In one embodiment the fermentation product comprises an enzyme selected from the group of enzyme classes consisting of oxidoreductases (EC 1), transferases (EC 2), hydrolases (EC 3), lyases (EC 4), isomerases (EC 5), and ligases (EC 6).

In another embodiment the enzyme is an enzyme with an activity selected from the group of enzyme activities consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, lysozyme, muramidase or xylanase.

Preferably the fermentation product is a protease or an alpha-amylase. Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 subdivisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

In some embodiments the fermentation products according to the invention include proteases such as Savinase, BPN', Subtilisin Carlsberg, TY145, 10R and variants of one of these having alterations, such as substitutions, insertions or deletions of amino acids, in 1, 2, 3, 4, 5, 6, 7, 8, 9 or 10 amino acid positions.

In some embodiments the fermentation product according to the invention is a protease having at least 60% sequence identity, at least 70%, at least 80%, at least 90%, at least 95%, at least 96%, at least 97%, at least 98% or at least 99% sequence identity to the sequence of SEQ ID NO: 1, SEQ ID NO: 2, SEQ ID NO: 3, SEQ ID NO: 4, SEQ ID NO: 5 or SEQ ID NO: 6.

The fermentation product may be an alpha-amylase or a glucoamylase and may be of bacterial or fungal origin. Protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO:

4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions: N128C+K178L+1182G+Y305R+G475K;

N128C+K178L+T182G+F202Y+Y305R+D3191+G475K;

S125A+N128C+K178L+T182G+Y305R+G475K; or

S125A+N128C+T1311+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, 1459S, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V1281 K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

In some embodiments the fermentation product according to the invention is an amylase having at least 60% sequence identity, at least 70%, at least 80%, at least 90%, at least 95%, at least 96%, at least 97%, at least 98% or at least 99% sequence identity to the sequence of SEQ ID NO: 7, SEQ ID NO: 8 or SEQ ID NO: 9.

The fermentation product may be obtained from a microorganism. The microorganism may be an organism that naturally produces the fermentation product or it may be generated using recombinant DNA technology where a gene encoding the desired fermentation product is operably linked with suitable control sequences such as promoter, terminator etc., suitable for expressing the gene in the intended host organism, and inserted into a suitable host organism. The microorganism may be a prokaryot or an eukaryot.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporis fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Pretreatments

Before the separation method according to the invention the fermentation broth may be subjected to one or more pretreatment steps, such as dilution, adjusting pH and/or temperature, adding stabilizers capable of preventing further growth of the microorganism, adding inhibitors capable of reducing protease activity and thereby limiting degradation due to proteolytic activity.

The pretreatment may also comprise a lysis step e.g. treating the broth with a chemical lysing agents such as one or more cell wall degrading enzymes e.g. lysozyme(s). Preferred lysis methods include adding lysozyme and/or hydrolysing enzymes to solubilise suspended material as cells or residual fermentation raw materials.

### First Separation Step

The fermentation broth comprising a desired fermentation product at least partially in solid form is according to the invention separated into at least two fraction in a first separation step, a first fraction comprising a liquid part of the fermentation broth comprising desired product in soluble form as well as other solubles such as salts and remaining soluble nutrients and it may comprise cells and cell debris; and a second fraction comprising the desired product in solid form, cells, cell debris and other insoluble material. The first phase generated in the first separation step may also be called the primary centrate or filtrate. The second phase may also be called the slurry or sludge phase.

Both the first fraction and the second fraction may comprise liquid part of the fermentation broth, cells and cell debris and desired product in solid form, but the ratios are completely different.

The first fraction comprises at least 60% of the liquid part of the fermentation broth, e.g. at least 70%, e.g. at least 80%, of the fermentation broth, whereas the second fraction comprises the reminder.

The second fraction comprises at least 60% of the desired product in solid form, preferably at least 65%, preferably at least 70%, preferably at least 75%, preferably at least 80%, preferably at least 85%, and most preferred at least 90%. The cells and cell debris is divided between the first and the second phase and the ratio depends on the particular separation technology used in the first separation step.

The first separation step is basically a separation step where solids are completely or partially separated from the liquid and the first separation step may therefore be performed using any technique known in the art for separating solids from a liquid phase. The first separation step may be done using filtration, decanting or centrifugation or any combination of these, and the separation may be done batch wise or continuously.

Depending on the selected separation technology used in the first separation step, it may be beneficial to add one or more Chemicals for assisting the separation before the separation. For example, one or more flocculants may be added before the first separation process as known in the art. The type and amount of the flocculants are selected based on the properties of the particular selected fermentation broth and will typically be determined using simple routing experimentation. This it known in the art and the selection of type and amount of one or more flocculants to facilitate the separation is completely within the skills of the skilled practitioner. For example may the methods disclosed in WO 2004/001054 be used according to the present invention.

### Solubilization Step.

The solubilisation step where desired product in the second phase is solubilized may be performed in any known method for solubilizing solid products, typically involving dilution with water or an aqueous medium, adding chemicals enhancing solubilisation and/or adjusting the pH.

The prior art discloses methods for solubilizing proteins, in particular proteins, in solid form in for example EP 2125865; U.S. Pat. No. 3,316,240 and WO 93/13125 and these methods are also useable according to the present invention.

In one embodiment the solubilisation step is performed by diluting the second phase with water or an aqueous media, optionally adding a solubilisation promoting chemical and reducing the pH.

In this embodiment the solubilisation may be done by diluting the second phase comprising desired product in solid form 100-2000% (w/w) based on the amount of the second phase, adjusting the pH to a pH value below 6.0, such as below 5.5, such as below 5.0, such as below 4.5; adding a solubilisation promoting chemical and mixing, whereby desired product is solubilized. The second phase comprising desired product in solid form is diluted 100-2000% (w/w), preferably 100-1500% (w/w), more preferred 100-1000% (w/w) and most preferred 200-700% (w/w).

The solubilsation promoting chemical may be any chemical promoting the solubilisation process e.g. a divalent salt, i.e. any salt comprising a divalent metal ion and soluble in the sludge phase; typically, a salt of Calcium, Magnesium, Ferric, Zinc salt comprising an anion selected among phosphates, sulphate, nitrate, acetate, chloride. Calcium or Magnesium salts are preferred. The solubilisation promoting chemical may be added at a concentration of 0.01-5% (w/w) based on the diluted second phase, preferably 0.01-1% (w/w) more preferred in the range of 0.1-0.5% (w/w).

The dilution medium will typically be water or an aqueous medium, such as an ultrafiltrate permeate or a condensate from an evaporation step recycled from another process performed at the same site such as in a subsequent step in the product recovery process.

The pH of the fermentation broth is in this embodiment adjusted to a pH value below pH 6.0, preferably below pH 5.5, in particular to a pH value below 5.0. The pH adjustment may be done before, simultaneously or after the addition of the divalent salt. The pH may be adjusted to a pH value between 2.0 and 5.5; preferably to a pH value between 2.0 and 5.0; more preferably to a pH value between 3.0 and 5.0, and in particular to a pH value between 4.0 and 5.0. It is to be noted that the process steps of the solubilisation step the dilution and the addition of a divalent salt and the pH adjustment may take place simultaneously or in any order. After adding the divalent salt and adjusting the pH, a mixing will take place. The mixing time will depend on the chosen temperature and the crystal morphology and/or structure of the protein in question. More than 80% of the crystals and/or precipitate and/or desired product bound to cell mass/insolubles may be solubilized according to the present invention; preferably more than 85%; more preferably more than 90% and in particular more than 95% of the crystals and/or precipitate and/or desired product bound to cell mass/insolubles may be solubilized

In another embodiment the solubilisation of the desired product in the second phase may be done by diluting the second phase with water or an aqueous solution and adjusting the pH to a high pH value, preferably to a pH value in the range of 9.5 to 13, such as the range of 10 to 13.

In still another embodiment the solubilisation or the desired product in the second phase is done by adding a chemical enhancing solubilisation of the desired product. The chemical enhancing solubilisation is preferably a polyol such as a low molecular weight polyethylene glycol, and the C₂ to C₈ alcohols having at least two OH groups, preferably with only two OH groups; especially preferred is the polyols where two OH groups are present on adjacent carbon atoms in the chain and the C₂-C₈ alcohol is aliphatic and have a straight carbon chain. Preferred polyols include ethylene glycol, propylene glycol, mono-propylene glycol, glycerol, the low molecular weight (about 900 or less) polyethylene glycols, and mixtures thereof.

In some embodiment an extended residence time is inserted after all ingredients and adjustments have been added in order to allow the dissolution of the desired product to take place. Like other chemical process the solubilisation of the desired product take time and a resident period may be beneficial to allow more product to dissolve. In principle the residence time may be extended until all the desired product is dissolved, but often the soluble product is more susceptible to degradation e.g. by proteases from the fermentation broth compared with the precipitated product, so in practice the skilled person often will select a relative short residence time in order to get as much desired product in solution but avoiding too much degradation of the soluble product.

The residence may typically be up to 12 hours, such as in the range of 0-500 minutes, preferably in the range of 15-300 minutes, more preferred in the range of 30-90 minutes.

After the solubilisation step the desired product may be recovered using methods known in the art such as further separation steps removing cells and cell debris and other solids from the mixture, concentration, formulation, drying such as spray drying, granulation etc.

In some applications the mixture is subjected to a lysis step after the solubilisation step such as treatment with a chemical lysing agents such as a cell wall degrading enzymes e.g. lysozyme. Preferred lysis methods include. adding lysozyme or hydrolysis enzymes to solubilise suspended material as cells or residual fermentation raw materials.

The particular selected process will be determined by several different factors such as the intended use of the desired product, available equipment and the amount of cells and cell debris in the second phase.

### Second Separation Step

In a preferred embodiment the method of the invention further comprises a second separation step removing cells and cell debris from the mixture comprising the solubilized desired product.

After the solubilisation step the mixture is subjected to the second separation step, which is basically a solid/liquid separation process removing solids, such as cells, cell debris and solids originating from the substrate; from the liquid fraction comprising the solubilized desired product. The second separation may be performed using any such equipment and methods known in the art for solid/liquid separations, such as centrifuges, decanters, filtration etc.

Depending on the selected separation technology used in the first separation step, it may be beneficial to add one or more Chemicals for assisting the separation before the separation. For example, one or more flocculants may be added before the first separation process as known in the art. The type and amount of the flocculants are selected based on the properties of the particular selected fermentation broth and will typically be determined using simple routing experimentation. This it known in the art and the selection of type and amount of one or more flocculants to facilitate the separation is completely within the skills of the skilled practitioner.

In one preferred embodiment, part or all of the first phase from the first separation is mixed with the mixture coming from the solubilisation step. In this way the two streams are combined and the desired product, both the part that was in solution in the fermentation broth and consequently contained in the first phase of the first separation step, and the part that was precipitated in the fermentation broth and consequently ended in the second phase of the first separation step, can be further purified and processed together in subsequent downstream operations.

This is particular beneficial for fermentations where the first phase comprises a significant amount of the desired product is solution. By mixing the first phase with the solubilized second phase before or after the second separation step will allow the skilled person to recover all desired product, both the part present in solid form in the second phase and the part present in solution in the first phase, together in downstream processes.

The mixing of the first phase of the first separation step and the stream from the solubilisation step comprising the solubilized desired product can take place either before or after the second separation step. If the first separation is performed in a way where the first phase connprises a significant amount of cell and cell debris is it preferred to add the first phase to the solubilized second phase before the second separation step so all cells and cell debris can be removed in the second separation step. If the first separation step is performed in a way so the first phase comprises no or only very minor amounts of the cells and cell debris; e.g. less than 1% of the total amount; the first phase may be added to the solubilized second phase either before or after the second separation step.

The skilled person will appreciate that the temperature may influence the process in various ways; the temperature may influence the solubilisation kinetics e.g. the solubilisation proceeds faster at a higher temperature; the temperature may also influence the stability of the desired product e.g. may the stability of a desired product decrease when the temperature rises. This means that for a given recovery process, the skilled person will have to optimize the temperature in order to obtain the best possible recovery process, and further it means that the temperature that is optimal for one fermentation product may not be optimal for another fermentation product. This is all known to the skilled person and can be done using typical routine experiments. The process of the invention is typically performed at a temperature in the range of 0-70° C., e.g. in the range of 10-50° C., often in the range of 15-45° C.

The method according to the invention has the benefit compared with prior art processes such as the method disclosed in EP 2 125 865 that the volume needed for solubilizing the desired product is significantly lower. Without wishing to be limited by any theory it is believed that this is because a substantial part of the salts present during fermentation where the precipitation took place has been removed with the first phase and is therefore absent during the solubilisation process. This has the benefit that the water consumption and /or chemicals enhancing solubilisation is significantly lower which has a number of significant additional benefits in industrial production scale operations, in addition to the cost of water and chemicals enhancing solubilisation; such as a reduced volume of waste water that need to be treated and further, the capacity costs for handling large volumes, equipment for downstream processing is reduced because the volumes that need to be treated are significantly lower.

Further, the solubilisation of a precipitated product according to the invention may also proceed faster that the solubilisation of the same product using a method according to prior art, giving the advantage of a higher throughput using the method according to the invention and consequently a lower demand for holding capacity.

### Subsequent Downstream Operations

The resulting desired product may be further isolated by methods known in the art. For example, the desired product may be recovered by conventional procedures including, but not limited to, further filtration, e.g., for removing any germs; ultra-filtration and micro-filtration, centrifugation, extraction, spray-drying, evaporation, precipitation or crystallization, hydrolysis (e.g. lysozyme or other) or other mechanical suspension degradation

### PREFERRED EMBODIMENTS

The invention may also be described by the following embodiments:

Embodiment 1: A method for recovering a desired product from a fermentation broth, where the desired product is present in precipitated form in the fermentation broth, the method comprising the steps of:
a) a first separation step separating the fermentation broth in a first phase and a second phase, wherein the first phase comprises supernatant, desired product in soluble form and optionally cells and cell debris, and the second phase comprises desired product in precipitated form, cells and cell debris; and
b) a solubilization step where the desired product in precipitated form in the second phase is solubilized.

Embodiment 2. The method according to embodiment 1, further comprising a second separation step where the solubilized desired product from step b) is separated from the cells and/or cell debris.

Embodiment 3. The method according to embodiment 1 or 2, wherein the first phase comprises at least 60% of the liquid part of the fermentation broth, e.g. at least 70%, e.g. at least 80%, e.g. at least 85%, e.g. at least 90% of the fermentation broth.

Embodiment 4. The method according to any of the embodiments 1-3, wherein the second fraction comprises at least 60% of the desired product in solid form, preferably at least 65%, e.g. at least 70%, e.g. at least 75%, e.g. at least 80%, e.g. at least 85%, and e.g. at least 99%.

Embodiment 5. The method according to any of the embodiments 1 to 4, wherein the desired product is selected among primary metabolites, secondary metabolites, proteins, vitamins, hormones and carbohydrates.

Embodiment 6. The method according to embodiment 5, wherein the desired product comprises one or more enzymes.

Embodiment 7. The method according to embodiment 6, wherein the one or more enzymes are selected from the group of enzyme classes consisting of oxidoreductases (EC 1), transferases (EC 2), hydrolases (EC 3), lyases (EC 4), isomerases (EC 5), and ligases (EC 6).

Embodiment 8. The method according to embodiment 7, wherein the one or more enzymes is/ are selected from the group of enzyme activities consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, lysozyme, muramidase or xylanase.

Embodiment 9. The method according to embodiment 8, wherein the one or more enzymes are selected among proteases.

Embodiment 10. The method according to embodiment 9, wherein the proteases are selected among subtilisins.

Embodiment 11. The method according to embodiment 10, wherein the proteases are selected among proteases having at least 80% sequence identity, such as at least 85% sequence identity, such as at least 95% sequence identity, such as at least 96% sequence identity, such as at least 97% sequence identity, such as at least 98% sequence identity, such as at least 99% sequence identity to one of SEQ ID NO: 1-6.

Embodiment 12. The method according to embodiment 11, wherein the protease is a variant of one of the proteases having the amino acid sequence of SEQ ID NO: 1-6, and variants of one of these having alterations, such as substitutions, insertions or deletions of amino acids, in 1, 2, 3, 4, 5, 6, 7, 8, 9 or 10 amino acid positions.

Embodiment 13. The method according to embodiment 8, wherein the one or more enzymes are selected among amylases.

Embodiment 14. The method according to embodiment 13, wherein the amylases are selected among alpha-amylases having at least 80% sequence identity, such as at least 85% sequence identity, such as at least 95% sequence identity, such as at least 96% sequence identity, such as at least 97% sequence identity, such as at least 98% sequence identity, such as at least 99% sequence identity to one of SEQ ID NO: 7-9.

Embodiment 15. The method according to embodiment 14, wherein the alpha-amylase is a variant of one of the polypeptides having the amino acid sequence of SEQ ID NO: 7-9, and variants of one of these having alterations, such as substitutions, insertions or deletions of amino acids, in 1, 2, 3, 4, 5, 6, 7, 8, 9 or 10 amino acid positions.

Embodiment 16. The method according to any of the preceding embodiments, wherein the desired product is obtained from a microorganism.

Embodiment 17 The method according to embodiment 16, wherein the microorganism is a prokaryot or an eukaryote.

Embodiment 18. The method according to embodiment 17, wherein the microorganism is a prokaryot selected among Bacillus, Clostridium, Enterococcus, Geobacillus, Lactobacillus, Lactococcus, Oceanobacillus, Staphylococcus, Streptococcus, Streptomyces, Campylobacter, E. coli, Flavobacterium, Fusobacterium, Helicobacter, Ilyobacter, Neisseria, Pseudomonas, Salmonella, and Ureaplasma.

Embodiment 19. The method according to embodiment 18, wherein the microorganism is a Bacillus cell selected among: Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

Embodiment 20. The method according to embodiment 17, wherein the microorganism is an eukaryote selected among Candida, Hansenula, Kluyveromyces, Pichia, Saccharomyces, Schizosaccharomyces, Yarrowia, Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

Embodiment 21. The method according to embodiment 20, wherein the microorganism is selected among: Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannico/a, Chrysosporium queenslandicum, Chrysosporium tropicum, Chrysosporium zonatum, Coprinus cinereus, Corio/us hirsutus, Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola inso/ens, Humicola /anuginosa, Mucor miehei, Myceliophthora thermophila, Neurospora crassa, Penicillium purpurogenum, Phanerochaete chrysosporium, Ph/ebia radiata, P/eurotus eryngii, Thielavia terrestris, Trametes villosa, Trametes versico/or, Trichoderma harzianum, Trichoderma koningii, Trichoderma longibrachiatum, Trichoderma reesei, or Trichoderma viride cell.

Embodiment 22. The method according to any of the preceding embodiments, wherein the fermentation broth has been fermented in a fermenter having a volume of at least 50 liters, such as at least 500 liters, such as at least 5,000 liters, such as at least 50,000 liters.

Embodiment 23. The method according to any of the preceding embodiments, wherein the first separation step is performed using centrifugation or filtration.

Embodiment 24. The method according to any of the preceding embodiments, wherein the solubilization step comprises:

i. Diluting the second phase 100-2000% (w/w) in water or an aqueous medium;

ii. adding a divalent salt; and

iii. adjusting the pH to a pH value below 6.0

Embodiment 25. The method according to embodiment 24, wherein the second phase is diluted 100-2000% (w/w), preferably 100-1500% (w/w), preferably 100-1000% (w/w) and most preferred 200-700% (w/w).

Embodiment 26. The method according to embodiment 24 or 25, wherein the second phase is diluted with water, tap water, an ultrafiltrate permeate or a condensate from an evaporation step.

Embodiment 27. The method according to any of the embodiments 24-26, wherein the divalent salt is selected among Calcium, Magnesium, Ferrous and Zinc salts comprising an anion selected among phosphates, sulphate, nitrate, acetate and chloride.

Embodiment 28. The method according to any of the embodiments 24-27, wherein the divalent salt is added in an amount of 0.01-5% (w/w) based on the diluted second phase, preferably 0.01-1% (w/w) more preferred in the range of 0.1-0.5% (w/w).

Embodiment 29. The method according to any of the embodiments 24-28, wherein the pH is adjusted to a pH value below a pH value below pH 6.0, preferably below pH 5.5, in particular to a pH value below 5.0. The pH adjustment may be done before, simultaneously or after the addition of the divalent salt. The pH may be adjusted to a pH value between 2.0 and 5.5; preferably to a pH value between 2.0 and 5.0; more preferably to a pH value between 3.0 and 5.0, and in particular to a pH value between 4.0 and 5.0.

Embodiment 30. The method according to any of the embodiments 1-23, wherein the solubilisation step is done by diluting the second phase with water or an aqueous solution and adjusting the pH to a pH value above 9.5, such as to a pH value in the range of 9.5 to 13, e.g. to a pH value in the range of 10 to 13.

Embodiment 31. The method according to any of the embodiments 1-23, wherein the solubilisation step is done by adding a chemical enhancing the solubilisation of the desired product. Embodiment 32. The method according to embodiment 31, wherein the chemical enhancing the solubilisation of the desired product is a polyol such as a low molecular weight polyethylene glycol or C₂ to C₆ alcohols having at least two OH groups, preferably with only two OH groups; especially preferred is the polyols where two OH groups are present on adjacent carbon atoms in the chain and the C₂-C₈ alcohol is aliphatic and have a straight carbon chain.

Embodiment 33. The method of embodiment 32, wherein the chemical enhancing solubilisation of the desired product is selected among ethylene glycol, propylene glycol, monopropylene, glycerol, polyethylene glycols having a molecular weight below 900 Da and mixtures thereof.

Embodiment 34. The method according to any of the embodiments 1-33, where the first separation step is performed by centrifugation or filtration.

Embodiment 35. The method according to embodiment 34, wherein the separation step is performed by filtration using a Drum filter.

Embodiment 36. The method according to embodiment 34, wherein the separation is performed by centrifugation using a continuous centrifuge, a decanter centrifuge etc.

Embodiment 37. The method according to any of embodiments 34 to 36, where a flocculant is added before the separation.

Embodiment 38. The method according to any of the preceding embodiments where the first phase from the first separation step is partly or completely added to the solubilized second phase.

Embodiment 39. The method according to embodiment 38, where the first phase from the first separation step is partly or completely added to the solubilized stream from step b) before the second separation step.

Embodiment 40. The method according to any of the previous embodiments comprising a pretreatment step before the first separation step selected among: dilution; adjusting pH and/or temperature, adding one or more stabilizers, adding one or more protease inhibitors.

Embodiment 41. The method according to any of the previous embodiments comprising one or more downstream operations after the second separation step, selected among: ultrafiltration, evaporation, concentration, stabilization, crystallization, spray drying and granulation.

### EXAMPLES

### Materials and Methods

Fermentation Broths for the examples below were provided from Novozymes NS, Kalundborg, Denmark. The fermentation broths were prepared by inoculating a Bacillus licheniformis strain transformed with a gene encoding the desired protease operationally connected with a promoter and regulatory sequences for expressing said gene; in an industrial fermenter in a fed-batch fermentation process. The fermentation process was terminated at the desired product concentration and the desired product was present in precipitated form at that time. By visual inspection using a microscope it could be clearly seen that the fermentation broth comprised crystalline material in addition to other solids including cells and cell debris.

In the examples below, the amount of solubilized product is defined as the ratio of product detected in the supernatant of a sample that has been subjected to a Relative Centrifugal Force of 2333 for 5 minutes, and the amount of product detected in an uncentrifuged sample.

Full dissolution is defined as occurring when the supernatant product concentration is within the measurement uncertainty of the concentration measured in an uncentrifuged sample. This value can be measured directly or calculated by extrapolation from multiple measurements.

The level of dilution of the fermentation broth in the given examples is used to reduce the concentration of components that are detrimental to solubility. The criteria for the level of dilution used in these examples is that it is high enough to solubilize all the product, but not in excess, to minimize water usage and process volumes.

The residence time for dissolution given in these examples is defined by process or experimental convenience and does not necessarily ensure full dissolution

### Example 1

### Recovery of Savinase

A fermentation broth from an industrial fermentation producing the protease having the amino acid sequence of SEQ ID NO: 1 (Savinase) was used in this example.

### A—Recovery According to Prior Art

Fermentation broth holding crystallized product enzyme was diluted 10 times with water (9L water added per kg Fermentation broth). Poly Aluminium Chloride and 34% (w/w) CaCl₂ was added (25 ml and 70 ml per kg Fermentation broth respectively) and the pH of the solution was adjusted to pH 4.2-4.6 using acetic acid. The mixture was given a residence time of 90 minutes at 41° C. whereby more that 80% of the product crystals were dissolved. The total volume was a little more than 10 times the initial volume of Fermentation broth.

This solution was then processed by a standard primary separation method: flocculation by cationic and anionic polymers and centrifugation to separate the solids from the liquid holding the dissolved product.

The liquid part was then processed further downstream in a series of steps for final product purification according to company specifications.

### B—Recovery According to the Invention

Fermentation broth holding crystallized product enzyme was diluted 3 times with water (2 kg water added per kg Fermentation broth) and separated according to the invention by centrifugation into a first phase (liquid phase) holding some cells and debris and a second phase (slurry) holding almost all the crystallized enzyme and some cells and debris. The separation was performed using a Custom designed centrifuge as further described in US 888939562. The first and second phases were both collected in separately. Approx. 0.75 kg second phase and 2.25 kg first phase was produced per kg Fermentation broth.

The second phase holding almost all the crystallized product was then diluted 12-13 times (11-12 kg water per kg second phase). Poly Aluminium Chloride and 34% (w/w) CaCl₂ was added (2.3 ml and 23 ml per kg second phase respectively) and the solution was pH adjusted to pH 4.2-4.6 using acetic acid. The mixture was given a residence time of 90 minutes at 41° C. whereby more that 80% of the crystals were dissolved.

The first phase from the first separation was then added to the diluted and solubilized second phase ending with a total volume of only a little more than 8 times the initial volume of culture broth. This clearly demonstrates reduced water consumption and process volumes using the method according to the invention.

This solution was then processed by a standard primary separation method: flocculation by cationic and anionic polymers and centrifugation to separate the solids from the liquid holding the dissolved product.

The liquid part was then processed further downstream in a series of steps for final product purification according to company specifications.

### Example 2

### Savinase Variant

A fermentation broth from an industrial fermentation producing a variant of the polypeptide having the amino acid sequence of SEQ ID NO: 1 with the substitutions: Y176A +R170S+A194P was used in this example. The product has precipitated and crystal were abundant in the fermentation broth.

### A—Recovery According to Prior Art.

Fermentation broth holding crystalized enzyme was diluted 14 times with water (13 kg water added per kg Fermentation broth). Poly Aluminium Chloride and 34% (w/w) CaCl₂ was added (25 ml and 80 ml per kg Fermentation broth respectively) and the solution was pH adjusted to pH 4.2-4.6 using acetic acid. The mixture was given a residence time of 90 minutes at 42° C. whereby more that 70% of the product crystals were dissolved. The total volume was a little more than 14 times the initial volume of Fermentation broth.

This solution was then processed by a standard primary separation method: flocculation by cationic and anionic polymers and centrifugation to separate the solids from the liquid holding the dissolved product.

The liquid part was then processed further downstream in a series of steps for final product purification according to company specifications.

### B—Recovery According to the Invention.

Fermentation broth holding crystallized product enzyme was diluted 3 times with water (2 kg water added per kg Fermentation broth) and separated according to the invention by centrifugation into a first phase (liquid phase) holding some cells and debris and a second phase (slurry) holding almost all the crystallized enzyme and some cells and debris. The separation was performed using a Custom designed centrifuge as further described in US 888939562. Approx. 0.5 kg second phase and 2 kg first phase was produced per kg Fermentation broth.

The second phase holding almost all the crystallized product was then diluted 14 times (13 kg water per kg second phase). Poly Aluminium Chloride and 34% (w/w) CaCl₂ was added (11.4 ml and 11 ml per kg second phase) and the solution was pH adjusted to pH 4.2-4.6 using acetic acid. The mixture was given a residence time of 90 minutes at 42° C. whereby more that 70% of the crystals were dissolved.

The total volume was only a little more than 7 times the initial volume of culture broth and clearly demonstrates reduced water consumption and process volumes using the method according to the invention.

This solution was then processed by a standard primary separation method: flocculation by cationic and anionic polymers and centrifugation to separate the solids from the liquid holding the dissolved product.

The liquid part was then processed further downstream in a series of steps for final product purification according to company specifications.

### Example 3

### Recovery of Savinase Via Drum Filtration.

A fermentation broth from an industrial fermentation producing the protease having the amino acid sequence of SEQ ID NO: 1 (Savinase) was used in this example.

### A—Recovery According to Prior Art

In this example, the fermentation broth holding crystallized product enzyme was diluted 10 times with tap water (9 kg water added per kg Fermentation broth), and then adjusted with 60m1 34% (w/w) CaCl₂ solution and 50 ml Poly Aluminium Chloride per kg fermentation broth. The pH was adjusted to pH 4.5 using acetic acid. The mixture was given a residence time 90 minutes at 42° C. whereby more that 80% of the crystals were dissolved. The total volume was a little more than 10 times the initial volume of Fermentation broth.

### B—Recovery According to the Invention

Fermentation broth holding crystallized product enzyme was diluted 3 times with tap water (2 kg water added per kg fermentation broth) and then adjusted with 20.0 ml Poly Aluminium Chloride per kg fermentation broth and pH was adjusted to pH 4.5 using acetic acid. 200 ml poly cationic polymer per kg fermented broth was added to aid separation by drum filtration into a first phase containing solubilized material and a second phase holding all the crystallized enzyme, cells and debris. The first and second phases were both collected in separate holding tank. Approximately 1 kg of second phase and 2 kg first phase was produced per kg Fermentation broth.

The second phase holding almost all the crystallized product, cells and cell debris was then diluted 3 times with tap water (2 kg water per kg second phase). A CaCl₂ (34% (w/w)) solution was added (18 mls per kg second phase) and pH was adjusted to pH 4.5 using acetic acid. The mixture was left standing in 60 minutes whereby more that 80% of the crystals were dissolved.

The first phase from the first separation was then added to the diluted and solubilized second phase ending with a total volume of a little more than 5 times the initial volume of the Fermentation broth and clearly demonstrating reduced water consumption and process volumes using the method according to the invention.

### Example 4

### Recovery of Amylase Via Centrifugation

A fermentation broth from an industrial fermentation producing an amylase variant prepared in Example 8 of WO 2001/066721 was used in this example.

### A—Recovery According to Prior Art

In this example, the fermentation broth holding crystallized product enzyme was diluted 20 times with tap water (19 kg water added per kg Fermentation broth), and then adjusted with 95 ml 34% (w/w) CaCl₂ solution and 30m1 Poly Aluminum Chloride per kg fermentation broth. The pH was adjusted to pH 5.0 using acetic acid. The mixture was given a residence time 20 minutes at 50° C. whereby more that 85% of the crystals were dissolved. The total volume was a little more than 20 times the initial volume of Fermentation broth.

### B—Recovery According to the Invention

Fermentation broth holding crystallized product enzyme was centrifuged for 5 mins at 2666 RCF. The upper half of the volume containing biomass and less than 5% of the product enzyme was removed and discarded. The remaining lower fraction containing more than 95% of the product enzyme was diluted 30 times with tap water (the equivalent of 29 kg water added per kg lower fraction) and then adjusted with 70 ml 34% (w/w) CaCl₂ solution and 30.0 ml Poly Aluminum Chloride per kg fermentation broth and pH was adjusted to pH 5.0 using acetic acid. The mixture was given a residence time 20 minutes at 50° C. whereby more that 90% of the crystals were dissolved. The total volume was a little more than 15 times the initial volume of Fermentation broth, clearly demonstrating reduced water consumption and process volumes using the method according to the invention.

### Example 5

### Recovery of Fungal Based Muramidase

Fermentation Broths for this example were provided from Pilot plant, Novozymes A/S, Bagsvaerd, Denmark. The fermentation broth was prepared by inoculating a fungal (Trichoderma reesei) strain transformed with a gene encoding the muramidase (SEQ ID NO: 4 in WO 2013/076253) operationally connected with a promoter and regulatory sequences for expressing said gene; in a large pilot-scale fermenter in a fed-batch fermentation process. By visual inspection using a microscope it could be clearly seen that the fermentation broth comprised crystalline material in addition to other solids including cells and cell debris.

The amount of solubilized product is in this example defined as the ratio of product detected in the supernatant of a sample that has been subjected to a Relative Centrifugal Force of 2333 for 5 minutes, and the amount of product detected in an uncentrifuged sample.

Full dissolution is defined as occurring when the supernatant product concentration is within the measurement uncertainty of the concentration measured in an uncentrifuged sample. This value can be measured directly or calculated by extrapolation from multiple measurements.

The level of dilution of the fermentation broth in the given examples is used to reduce the concentration of components that are detrimental to solubility. The criteria for the level of dilution used in these examples is that it is high enough to solubilize all the product, but not in excess, to minimize water usage and process volumes.

The residence time for dissolution given in these examples is defined by process or experimental convenience and does not necessarily ensure full dissolution.

### A—Recovery According to Prior Art

Fermentation broth holding crystalized enzyme was diluted 7 times with process water (6 kg water added per kg Fermentation broth). Poly Aluminium Chloride was added (15 ml per kg Fermentation broth) and the solution was pH adjusted using phosphoric acid. The mixture was given a residence time of 120 minutes at 25° C. whereby more that 75% of the product crystals were dissolved. The total volume was a little more than 7 times the initial volume of Fermentation broth.

This solution was then processed by a standard primary separation method: flocculation by cationic and anionic polymers and centrifugation to separate the solids from the liquid holding the dissolved product.

The liquid part was then processed further downstream in a series of steps for final product purification according to company specifications.

### B—Recovery According to the Invention

Fermentation broth holding crystallized product enzyme was diluted 2,5 times with process water (1.5 kg water added per kg Fermentation broth) and separated according to the invention by centrifugation into a first phase (liquid phase) holding some cells and debris and a second phase (slurry) holding almost all the crystallized enzyme and some cells and debris. The separation was performed using a SB7 (Westfalia) discharge centrifuge. The first and second phases were both collected separately. Approx. 0.55 kg second phase and 1,95 kg first phase was produced per kg Fermentation broth.

The second phase holding almost all the crystallized product was then treated by exactly the same method as regular Culture broth; diluted 7 times (6 kg process water per kg second phase). Poly Aluminium Chloride was added (15 ml per kg second phase) and the solution was pH adjusted using phosphoric acid. This mixture was just given a residence time of 60 minutes at 25° C. whereby more that 85% of the crystals were dissolved.

The first phase from the first separation was then added to the diluted and solubilized second phase ending with a total volume of approx. 6 times the initial volume of culture broth. This demonstrates reduced water consumption and process volumes as well as shorter dissolution time (higher dissolved product yield) using the method according to the invention.

This solution was then processed by a standard primary separation method: flocculation by cationic and anionic polymers and centrifugation to separate the solids from the liquid holding the dissolved product.

The liquid part was then processed further downstream in a series of steps for final product purification according to company specifications.

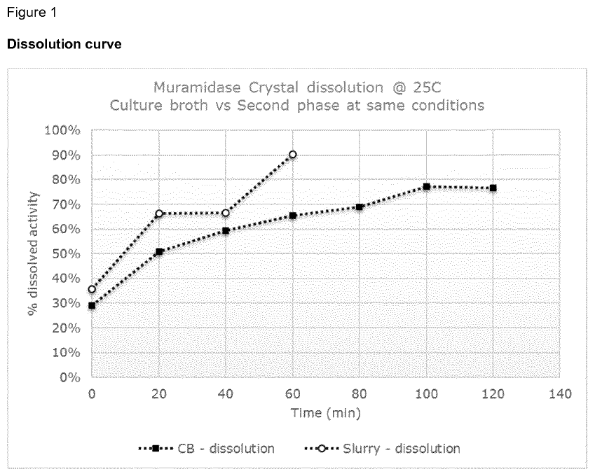

The dissolution curves for the culture broth (CB) according to part A, and the slurry found in the second phase according to part B are shown in FIG. 1, and it shown clearly that the dissolution proceeded faster using the method according to the present invention, compared with the traditional method. The figure also shows that a higher dissolution was obtained after 60 minutes according to the invention compared with after 120 minutes using the procedure of the prior art.

1. A method for recovering a desired product from a fermentation broth, where the desired product is present in precipitated form in the fermentation broth, comprising the steps of:
a) a first separation step separating the fermentation broth in a first phase and a second phase, wherein the first phase comprises supernatant, desired product in soluble form and optionally cells and cell debris, and the second phase comprises desired product in precipitated form, cells and cell debris; and
b) a solubilization step where the desired product in precipitated form in the second phase is solubilized.

2. The method according to claim 1, further comprising a second separation step where the solubilized desired product from step b) is separated from the cells or cell debris.

3. The method according to claim 1, wherein the first phase comprises at least 60% of the liquid part of the fermentation broth.

4. The method according to claim 1, wherein the second phase comprises at least 60% of the desired product in solid form.

5. The method according to claim 1, wherein the desired product comprises one or more enzymes.

6. The method according to claim 5, wherein the one or more enzymes are selected from the group of enzyme activities consisting of aminopeptidase, amylase, amyloglucosidase, mannanase, carbohydrase, carboxypeptidase, catalase, cellulase, chitinase, cutinase, cyclodextrin glycosyltransferase, deoxyribonuclease, esterase, galactosidase, beta-galactosidase, glucoamylase, glucose oxidase, glucosidase, haloperoxidase, hemicellulase, invertase, isomerase, laccase, ligase, lipase, lyase, mannosidase, oxidase, pectinase, peroxidase, phytase, phenoloxidase, polyphenoloxidase, protease, ribonuclease, transferase, transglutaminase, lysozyme, muramidase and xylanase.

7. The method according to claim 6, wherein the one or more enzymes are proteases having at least 80% sequence identity to one of SEQ ID NOs: 1-6.

8. The method according to claim 6, wherein the one or more enzymes are amylases having at least 80% sequence identity, to one of SEQ ID NOs: 7-9.

9. The method according to claim 1, wherein the desired product is obtained from a microorganism.

10. The method according to claim 9, wherein the microorganism is a prokaryote selected from the group consisting of Bacillus, Clostridium, Enterococcus, Geobacillus, Lactobacillus, Lactococcus, Oceanobacillus, Staphylococcus, Streptococcus, Streptomyces, Campylobacter, E. coli, Flavobacterium, Fusobacterium, Helicobacter, Ilyobacter, Neisseria, Pseudomonas, Salmonella, and Ureaplasma.

11. The method according to claim 10, wherein the microorganism is a Bacillus selected from the group consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis.

12. The method according to claim 9, wherein the microorganism is a eukaryote selected from the group consisting of Candida, Hansenula, Kluyveromyces, Pichia, Saccharomyces, Schizosaccharomyces, Yarrowia, Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phiebia, Piromyces, Pleurotus, Schizophyllum, Talaromyces, Thermoascus, Thielavia, Tolypocladium, Trametes, and Trichoderma.

13. The method according to claim 12, wherein the microorganism is selected from the group consisting of Kluyveromyces lactis, Saccharomyces carisbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium suiphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phiebia radiata, Pleurotus eryngii, Thielavia terrestris, Trametes villosa, Trametes versicolor, Trichoderma harzianum, Trichoderma koningii Trichoderma longibrachiatum, Trichoderma reesei, and Trichoderma viride.

14. The method according to claim 1, wherein the first separation step is performed using centrifugation or filtration.

15. The method according to claim 1, wherein the solubilization step comprises:
i. diluting the second phase 100-2000% (w/w) in water or an aqueous medium;
ii. adding a divalent salt; and
iii. adjusting the pH to a value below 6.0.

16. The method according to claim 15, wherein the second phase is diluted 100-1500% (w/w).

17. The method according to claim 15, wherein the divalent salt is selected from the group consisting of calcium, magnesium, ferrous and zinc salts, comprising an anion selected from the group consisting of phosphates, sulphate, nitrate, acetate and chloride, and is added in an amount of 0.01-5% (w/w) based on the diluted second phase.

18. The method according to claim 15, wherein the pH is adjusted to a value below pH 5.5.

19. The method according to claim 1, wherein the solubilization step is done by diluting the second phase with water or an aqueous solution and adjusting the pH to a value above 9.5.

20. The method according to claim 1, wherein the solubilisation step is done by adding a chemical enhancing the solubilization of the desired product.

21. The method according to claim 20, wherein the chemical enhancing the solubilization of the desired product is a polyol, including a low molecular weight polyethylene glycol or C₂ to C₈ alcohol having at least two OH groups. .

22. The method according to claim 1, where the first phase from the first separation step is partly or completely added to the solubilized second phase.

23. The method according to claim 1, comprising a pretreatment step before the first separation step selected from the group consisting of dilution, adjusting pH or temperature, adding one or more stabilizers, and adding one or more protease inhibitors.

24. The method according to claim 1, comprising one or more downstream operations after the second separation step, selected from the group consisting of ultrafiltration, evaporation, concentration, stabilization, crystallization, spray drying and granulation.


SEQUENCE LISTING (9 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 275 aa, Bacillus amyloliquefaciens [sequence omitted]

SEQ ID NO: 3 - protein, 274 aa, Bacillus sp.
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 4 - protein, 311 aa, Bacillus sp. [sequence omitted]

SEQ ID NO: 5 - protein, 188 aa, Nocardiopsis sp.
ADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 6 - protein, 412 aa, Pyrococcus furiosus
AELEGLDESAAQVMATYVWNLGYDGSGITIGIIDTGIDASHPDLQGKVIGWVDFVNGRSYPYDDHGHGTHVASIAAGTGAASNGKYKGMAPGAKLAGIKVLGADGSGSISTIIKGVEWAVDNKDKYGIKVINLSLGSSQSSDGTDALSQAVNAAWDAGLVVVVAAGNSGPNKYTIGSPAAASKVITVGAVDKYDVITSFSSRGPTADGRLKPEVVAPGNWIIAARASGTSMGQPINDYYTAAPGTSMATPHVAGIAALLLQAHPSWTPDKVKTALIETADIVKPDEIADIAYGAGRVNAYKAINYDNYAKLVFTGYVANKGSQTHQFVISGASFVTATLYWDNANSDLDLYLYDPNGNQVDYSYTAYYGFEKVGYYNPTDGTWTIKVVSYSGSANYQVDVVSDGSLSQPGSS

SEQ ID NO: 7 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 8 - protein, 514 aa, Bacillus stearothermophilus
APFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 9 - protein, 483 aa, Bacillus licheniformis [sequence omitted]